(19) United States
(12) Patent Application Publication
(10) Pub. No.: US 2022/0061968 A1
(43) Pub. Date: Mar. 3, 2022

(54) Dental Prosthesis

(71) Applicant: Ivoclar Vivadent AG, Schaan (LI)

(72) Inventors: Thomas Baaske, Mols (CH); Antonio Ferilli, Wängi (CH)

(21) Appl. No.: 17/380,082

(22) Filed: Jul. 20, 2021

(30) Foreign Application Priority Data
Sep. 2, 2020 (EP) 20194142.4

(51) Int. Cl. A61C 13/277; A61C 13/08; A61C 13/01; A61C 8/00; A61C 5/30; A61C 13/00; A61K 6/889

(52) U.S. Cl. CPC A61C 13/277; A61C 13/082; A61C 13/01; A61C 8/0095; A61C 13/0022; A61C 5/30; A61C 13/0004; A61K 6/889; A61C 13/0019; A61C 8/005

(57) ABSTRACT

In the case of a dental prosthesis comprising a gum-coloured prosthesis base and a tooth coloured tooth arrangement which is partially embedded into the prosthesis base, the dental prosthesis is formed as a hybrid prosthesis, wherein the prosthesis base has a contact surface which has a surface normal pointing towards the gums.

The tooth arrangement (14) surrounds an external telescope (secondary part) or an abutment on at least 2 sides (26, 28, 30), is formed as a one-piece tooth quadrant or as a segmented tooth quadrant (16) and consists of a harder material than the prosthesis base (12).

### CROSS-REFERENCE TO RELATED APPLICATIONS

This application claims priority to European patent application No. 20194142.4 filed on Sep. 2, 2020, the disclosure of which is incorporated herein by reference in its entirety.

### TECHNICAL FIELD

The invention relates to a dental prosthesis.

### BACKGROUND

Dental prostheses consist typically of a gum-coloured prosthesis base and a tooth-colored tooth arrangement. They are fixedly connected to one another, wherein the prosthesis base grasps and holds the tooth arrangement, and wherein typically the tooth arrangement is partially embedded into the prosthesis base. Moreover, the prosthesis base is used for contact and anchoring on or at the alveolar crest.

By reason of the chewing movements and the alternating loads associated therewith, dental prostheses are subjected to particular loads specifically at their point of contact or anchoring.

Typically, at least two implants are anchored in the jawbone and anchoring is ensured by means of so-called primary structures and secondary structures.

The anchoring is effected between an implant, optionally with an abutment, with a male part which is also defined as a primary structure or as an internal telescope, and a female part, attached to the dental prosthesis, which is also defined as a secondary structure or external telescope.

Since this anchoring has to transfer considerable forces towards the prosthesis, a typically metal reinforcing framework is used as a tertiary structure which surrounds the secondary structure and is intended to ensure better force distribution into the prosthesis and towards the tooth arrangement.

It has also been proposed to anchor the tooth arrangement to the tertiary structure, especially if it has individual teeth. However, this solution is comparatively complex and requires the height position of the teeth to be precisely fixed in advance.

Furthermore, so-called hybrid prostheses have become known as a special form of dental prostheses. A hybrid prosthesis can be a type of prosthesis that is supported by dental implants, that is, an implant-supported prosthesis. The implant-supported prosthesis consists of a prosthetic structure anchored by implants that have been previously placed inside the maxillary bones, in order to recover function and aesthetics as much as possible.

They permit better force distribution. Apart from the anchoring to the primary, secondary and tertiary structures via the implants, the prosthesis base in the case of a hybrid prosthesis has at least two contact surfaces.

They are precisely adapted to the patient's alveolar crest and are able to absorb considerable forces in a pain-free manner.

In a mouth without any teeth, a plurality of contact surfaces can extend in a manner distributed over the entire alveolar crest, or can even form one large contact surface overall. This contact surface can be formed in the manner of a tooth quadrant in the form of a channel which has a U-shaped cross-section so that it can absorb not only occlusal forces but also obliquely impacting and horizontal forces arising from the chewing movement.

Such hybrid prostheses are known e.g. from FR 2 593 058 A1 and U.S. Pat. No. 4,657,510 A1, which U.S. patent is hereby incorporated by reference in its entirety. In the case of the aforementioned U.S. Pat. No. 4,657,510 A1, each tooth is supported by means of a special implant system. Although this offers effective anchoring, it is not always applicable and also requires a plurality of jawbone bores.

In the case of the solution according to FR 2 593 058 A1, effective anchoring is to be achieved between the prosthesis and the primary structure and the interchangeability is to be provided.

Furthermore, it has been proposed to design hybrid prostheses in such a manner that a limited number of primary structures is produced as an implant construction part, which are supported on implants, e.g. 2, 3 or 4, in the region of the incisors and premolars or optionally a respective one in the region of the molars, and as seen distally from the rearmost implant an enlarged contact surface is produced which is to serve to provide improved support at this location.

This solution offers an effective compromise in terms of the absorption and distribution of chewing forces. However, it is comparatively expensive and inflexible in terms of the tertiary structure which is to be embedded in a complex manner.

### SUMMARY OF THE INVENTION

In contrast thereto, the object of the invention is to provide a dental prosthesis according to the claims, which can be handled flexibly and which offers, at the same or lower production cost, at least the same long-term durability coupled with good wear comfort.

In accordance with the invention, this object is achieved by the independent claim(s). Advantageous developments are apparent from the dependent claims.

In accordance with the invention, provision is made to produce the tooth arrangement as a one-piece full tooth quadrant or as a segmented tooth quadrant.

On the one hand, such a segmented tooth quadrant which can also be defined as a tooth quadrant segment permits efficient manufacture by means of CAD/CAM. On the other hand, it has been demonstrated that a dental prosthesis which is constructed from or has segmented or one-piece tooth quadrants instead of individual teeth has considerably improved bending strength and also bending tensile strength.

In comparison with the production of prostheses with individual teeth, the bending strength can be twice as great.

In accordance with the invention, it is preferred that the tooth quadrant consists of a harder material than the prosthesis base, which ensures the aforementioned property.

In accordance with the invention, provision is made that the tooth arrangement as a tooth quadrant is extended towards the gums in comparison with the hitherto known hybrid prostheses. The tooth quadrant forms, on its basal side, a bearing element for bearing the prosthesis.

The bearing element receives a secondary part and surrounds it. The secondary part can be formed e.g. as an external telescope, a locator, a ball anchor, a conical crown or any other retention part. US 20100248186, 20070111163, 20070020582, 20030123943, U.S. Pat. Nos. 10,543,068, 10,617,497, 9,855,120, 8,678,822, and 6,832,877, each disclose an example of a retention means and are hereby incorporated by reference in their entirety. Other examples of implant overdentures attachment systems having two parts include the matrix being the receptacle component, and a corresponding patrix that fits closely within the matrix, either mechanically, or with a friction fit. One part of the attachment system is usually connected to the implant and the other incorporated within the undersurface of the overdenture. The patrix designs take the form of a connecting bar/s or free-standing ball/magnetic abutments. The matrix designs are adjustable and/or replaceable, are mechanical, friction fit or magnetic, and usually take the form of clips for bars; metal lamellae; plastic/nylon caps springs, or rubber O-rings for ball abutments; keepers or retainers for magnetic abutments. The range of available attachment systems permits dentists and dental technicians to select a retentive mechanism that reflects personal experience and preference, plus necessary patient considerations.

The secondary part interacts with the primary part in a manner known per se. The primary part can be formed e.g. by an abutment or a tooth stump, or else can be formed in each case by a part thereof.

Preferably, the tooth quadrant in accordance with the invention is extended towards the gums. In accordance with the invention, the tooth quadrant prolongation towards the gums simulates a tertiary structure.

In accordance with the invention, the tertiary structure is dispensable. The prosthesis in accordance with the invention is free of tertiary structures. However, this also ensures that no reinforcing elements, e.g. metal elements, have to be embedded into the prosthesis base.

Therefore, the prosthesis base can likewise be produced more easily by milling. It is particularly favourable if the tooth arrangement extends towards the gums as far as the secondary structure, surrounds and receives said structure and is thus used for anchoring purposes and to transfer chewing forces. It then has direct contact with the secondary structure or contact via a thin layer of base material.

Preferably, the contact is not merely a touch-contact with the secondary structure but instead is at least partial engagement around said structure. In order to ensure such engaging contact around said structure, the tooth arrangement or the tooth quadrant is channel-shaped on its gum side or basal side, corresponding to the course of the tooth quadrant, or has at least suitable fitting apertures at the points, at which a secondary structure is to be introduced. The tooth arrangement forms at this location, i.e. on its basal side, a bearing element for bearing the secondary structure.

It is connected in a suitable manner to the bearing element. This connection can be established by incorporation by polymerisation or by means of an adhesive.

In an advantageous embodiment, the tooth quadrant has direct contact with the secondary structure on its lower side, i.e. basal side or gum side. This permits secure anchoring and effective force transfer. This applies in particular when the secondary part is received completely in the lower region or basal region of the tooth quadrant. The terms secondary part and secondary structure are used here synonymously.

In a modified embodiment, provision is made that the tooth quadrant is suitably shaped in its basal region in order to receive the secondary structure. In the case of this embodiment, the tooth quadrant is covered by a thin layer of base material which, for its part, receives the secondary part. In this case, an adhesive is preferably provided which fixes the secondary structure at this location.

This solution permits a slightly more elastic accommodation of the secondary part in the tooth quadrant without the accommodation being adversely affected specifically also by shearing forces.

In the preferred embodiments of the invention, the tooth segments of the tooth quadrant or the tooth quadrant is/are configured such that for each segment or each tooth quadrant at least two secondary parts are used for anchoring purposes. This permits particularly bending-resistant and connecting-resistant anchoring of the tooth quadrant to the jawbone.

Preferably, the tooth quadrant or the tooth segment is configured such that corresponding apertures are present in the basal region at the points at which secondary structures are provided.

For the remainder, the basal region is filled with the tooth quadrant material and is covered by prosthesis base material at this location.

In a modified embodiment, a continuous channel is provided which is filled with the secondary structure at the points at which said secondary structure is provided. In contrast, the channel is then filled with prosthesis base materials at the points outside the secondary structures.

Whilst the dental prosthesis in accordance with the invention is described herein in conjunction with a telescope bearing having an internal telescope=primary part and an external telescope=secondary part, it is understood that, instead of this, it is possible to have a bearing on an abutment which, for its part, is anchored on the implant. Alternatively, it is also possible to have the bearing on a tooth stump.

The abutment then extends at the point, at which the secondary part is provided, and in the same manner a secure bearing and anchoring is possible on the tooth quadrant material, optionally via a thin layer of prosthesis base material.

The required apertures can be produced in the tooth quadrant material as a matter of course because in any case the dental prosthesis is manufactured preferably by machining methods such as by milling or constructive methods.

With regard to the connection between the prosthesis base and tooth arrangement, it is preferred that both elements are manufactured from separate blanks. The tooth quadrant is provided at the contact points to the prosthesis base with a self-polymerising adhesive in a manner known per se, is inserted into the tooth quadrant aperture of the prosthesis base and then polymerises automatically.

In the same manner, after the connection is established between the prosthesis base and tooth arrangement, the secondary structure can be inserted into the designated apertures or the designated channel, wherein good adhesion is ensured with a corresponding adhesive.

It is also possible to provide the secondary structure with small apertures and to produce corresponding protrusions on the inside of the bearing element of the tooth arrangement. Upon being slid on, the protrusions then snap into the apertures, which further improves the fixing and bearing of the tooth arrangement on the secondary structure. In this respect, it is also possible to have a purely interlocking bearing or optionally—by means of adhesive—a combined adhesive and interlocking connection.

When implementing a snap-latching connection, it is also possible to produce this between the secondary part and the associated aperture in the tooth arrangement and then initially latch the secondary part at this location.

The lower flanks of the basal region of the tooth quadrant are comparatively flexible, which facilitates latching. In the case of this solution, provision is made, initially following on from this, to glue the combination of the secondary part and tooth quadrant into the prosthesis base material and thus provide stiffening.

In accordance with the invention, provision is made to completely dispense with a tertiary structure. Therefore, it is not necessary to provide a reinforcing structure, e.g. a metal inclusion, in the prosthesis base and the costly steps required for this purpose can be omitted.

The dental prosthesis herein can connect directly to a secondary part and/or a primary part or directly to an abutment and/or implant structure without the need for a tertiary structure or reinforcing structure or metal inclusion structure.

If at least the tooth arrangement is produced by CAD/CAM, i.e. in general by milling, provision is made to store a virtual form of the basal region of the tooth arrangement which then always matches the associated secondary structure.

In this case, edge-free transitions are preferred. When making diameter adjustments between the tooth root and the basal region of the tooth which is characterised by the U-shaped or channel-shaped configuration, it is preferred to produce soft transitions, e.g. S-shapes.

It is understood that, instead of this, the tooth root quickly becomes spread or narrower so that it matches the U-shaped basal region of the tooth quadrant in accordance with the invention.

Furthermore, it is also possible to configure the connection of the basal region to the alveolar crest in an arbitrary manner. However, if, in the first embodiment, the basal region of the tooth consisting of the harder tooth quadrant material lies directly against the secondary structure, the part of the basal region facing towards the alveolar crest can still be protected by a thin layer of prosthesis base material. This solution is preferred.

Alternatively, it is also possible to pull the tooth quadrant material at this location so far down that contact is established between the alveolar crest and the tooth quadrant material.

It is also possible to have the tooth quadrant material terminate in advance of the alveolar crest such that a layer consisting of base material extends between the alveolar crest and the tooth quadrant and the prosthesis base material completely covers the alveolar crest.

In an advantageous embodiment, provision is made that the tooth arrangement is formed by the complete tooth quadrant. In this embodiment, the tooth arrangement is particularly bending-resistant since the tooth material has a high bending strength.

It is understood that, if required, the tooth arrangement can be in any suitable configuration in the basal region outside the points, at which the secondary structures and the contact surfaces are provided. For example, in order to increase the stiffness, it is possible to provide ribs or protrusions which can increase the bending strength and bending tensile strength of the tooth quadrant per se and thus also of the dental prosthesis on the whole.

In a modified embodiment, provision is made to provide the basal region of the tooth quadrant at the points discussed here with a type of rib/lattice structure and to form the tooth quadrant by means of a machining method, such as by milling on a CAD/CAM device, and to form the prosthesis base by means of an applying method, such as e.g. by rapid prototyping directly on the tooth quadrant at the appropriate location.

A correspondingly high-quality dental prosthesis can also be sprayed on by injection-moulding, wherein the injection mould is then preferably likewise produced by rapid prototyping.

The type and arrangement of the secondary structures and the contact surfaces can be adapted to the requirements within wide ranges. For example, in each case a secondary structure can be provided in the canine region and a contact surface can be provided at the distal end of the prosthesis.

This solution has the advantage of statically determined fixing of the dental prosthesis on the alveolar crest and at the same time the advantage of gentle force distribution on large contact surfaces which increase wear comfort.

When producing the tooth arrangement from segments of the entire tooth quadrant, i.e. from partial tooth quadrants, it is also possible to produce contact surfaces in the transition region between partial segment quadrants. In the case of this solution, a secondary structure which is used for anchoring purposes is then preferably provided for each tooth segment. In this way, force is applied in an alternating manner via secondary structures into the patient's jawbone and via the contact surface on the alveolar crest.

In accordance with the invention, it is essential that metal inclusions or attachments of the hybrid prosthesis can be completely dispensed with. A tertiary structure does not have to be manufactured or attached in order to provide the desired force transfer and stability.

It is preferable that the dental prosthesis includes a gum-coloured prosthesis base and a tooth-coloured tooth arrangement, which tooth-coloured tooth arrangement is partially embedded into the prosthesis base, the dental prosthesis formed as a hybrid prosthesis, the prosthesis base having a contact surface which has a surface normal pointing towards the gums, the tooth arrangement surrounding a secondary part or a primary part on at least two sides and formed as a one-piece tooth quadrant or as a segmented tooth quadrant.

It is preferable that the secondary part or the primary part supports the tooth arrangement and a layer of the base material lies therebetween.

It is preferable that the tooth-coloured tooth material of the tooth arrangement is fabricated of a material having a greater hardness than the hardness of the prosthesis material of the prosthesis base.

It is preferable that the secondary part is formed having an outer or external telescope configuration and/or the primary part is formed as an abutment or as a tooth stump, which fits within the external telescope configuration.

It is preferable that the tooth arrangement is produced by a machining or constructive method.

It is preferable that the tooth arrangement surrounds, on its basal side, the external telescope configuration or the primary part in the oral, vestibular and occlusal directions.

It is preferable that the tooth arrangement forms, on its basal side, which is the side facing the gum, a bearing element which receives the secondary part or the primary part, optionally via a layer of base material.

It is preferable that the bearing element of the tooth arrangement is embedded towards the outside in the vestibular and oral directions, into the prosthesis base material which supports the bearing element from the outside, and the bearing element is exposed and open, on the inside towards the gums.

It is preferable that the tooth arrangement is fabricated at least partially of a highly cross-linked polymethyl methacrylate (PMMA), and the prosthesis base is fabricated of a less highly cross-linked PMMA in comparison with the highly cross-linked PMMA material used for the tooth arrangement.

It is preferable that the tooth arrangement is constructed of two layers or multiple layers of dentine material and of enamel material which enamel material is more translucent than the dentine material and which dentine material extends in one piece to the bearing element and to form the bearing element and that the highly cross-linked PMMA comprises a double cross-linked (DCL) material.

It is preferable that the tooth arrangement has segments of at least 3 teeth which are fixedly connected to one another and to the bearing element via dentine material. Tooth arrangements can contain 1, 2, 3, 4, 5, 6 and so on up to 10 or 12 or 14 for a full denture.

It is preferable that the dental prosthesis is metal-free and is produced by machining or constructive methods.

It is preferable that the bearing element is formed from tooth material in an arcuate manner in plan view, corresponding to the tooth quadrant, and in a U-shape in cross-section, for the lower jaw prosthesis in the form of an upside-down U.

It is preferable that the tooth material extends to the contact surface of the prosthesis base and is covered by base material forming said contact surface.

It is preferable that the tooth material is covered by a layer of base material, in a layer thickness of at least 0.5 mm or at least about 0.8 mm.

It is preferable that the tooth quadrant and the prosthesis base are each produced separately by milling from blanks or by means of an additive method and are fixedly connected to one another by an adhesive and that the adhesive is a self-polymerising adhesive.

### BRIEF DESCRIPTION OF THE DRAWINGS

Further details, advantages and features will be apparent from the following description of several exemplified embodiments of the invention with reference to the drawing.

In the drawings:

FIG. 1 shows a schematic perspective view of a dental prosthesis as a hybrid prosthesis in one embodiment of the invention;

FIG. 2 shows a sectional view of a dental prosthesis in accordance with the invention in a further embodiment;

FIG. 3 shows a sectional view of a dental prosthesis in a further embodiment;

FIG. 4 shows a sectional view of a dental prosthesis in a further embodiment;

FIG. 5 shows a sectional view of a dental prosthesis in a further embodiment;

FIG. 6 shows a sectional view of a dental prosthesis in a further embodiment; and

FIG. 7 shows a sectional view of a dental prosthesis in a further embodiment.

### DETAILED DESCRIPTION

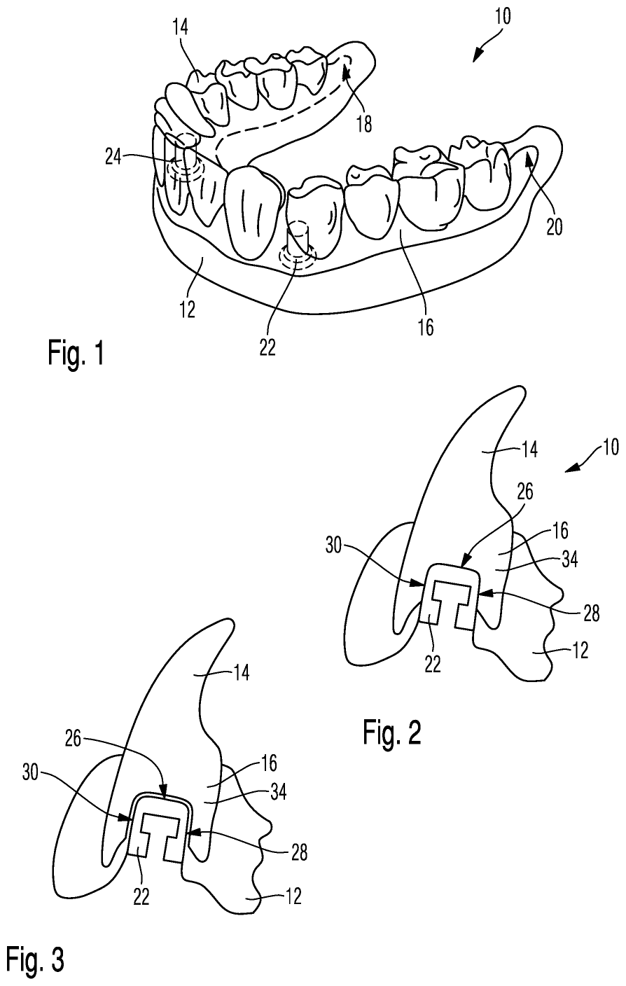

In all of the embodiments of the invention, the dental prosthesis 10 illustrated in FIG. 1 consists, in a manner known per se, of a prosthesis base 12 and a tooth arrangement 14, wherein, likewise in a manner known per se, the prosthesis base 12 is gum-coloured and the tooth arrangement 14 is tooth-coloured.

The tooth arrangement 14 is constructed on a common tooth base 34 as a tooth quadrant, in one piece in the illustrated exemplified embodiment. In this way, the common tooth base 34 connects the tooth arrangement in one piece.

The prosthesis base 12 receives the tooth base completely, wherein, in a manner known per se, the prosthesis base material 12 is pulled up in the region of the tooth transitions, which gives the appearance of individual teeth.

The tooth quadrant 16 consists of a harder material than the prosthesis base 12, e.g. highly cross-linked polymethyl methacrylate (PMMA).

The prosthesis base 12 has two contact surfaces 18 and 20 which point downwards or upwards (depending on the upper or lower jaw) with their surface normals and are intended to lie on the patient's alveolar crest 42. They are formed preferably at the distal end of the prosthesis 10.

In this case, surface normal is to be understood to mean a suitable surface normal which points downwards (in the case of a lower jaw and upwards in the case of an upper jaw) such that the surface can serve as a contact surface 18. In fact, the contact surfaces 18 and 20 are curved, corresponding to the alveolar crest 42 formed at this point.

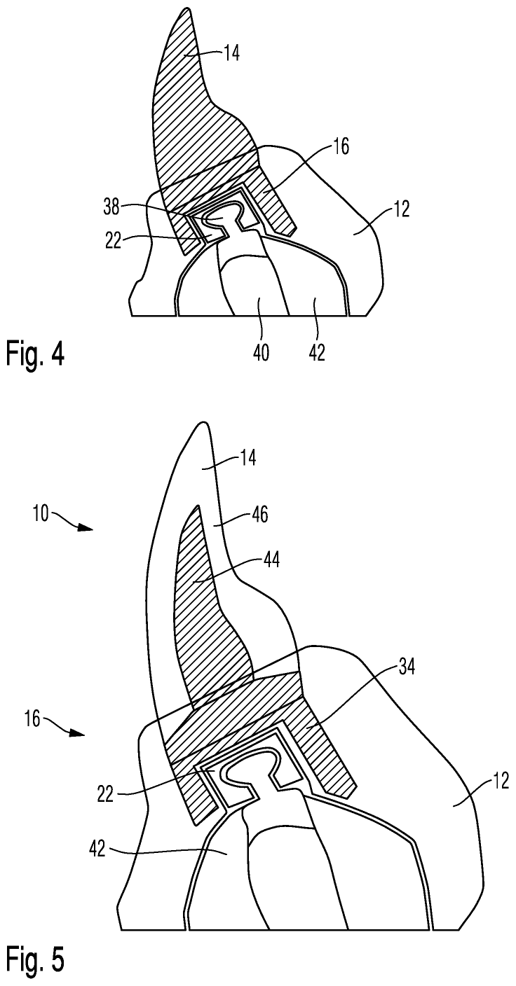

Furthermore, in order to anchor the dental prosthesis 10 to the patient's jaw two schematically illustrated secondary structures 22 and 24 are provided. They are formed in a manner known per se as an outer telescope or female fitting or socket or receptacle part and rest on a primary structure 38, (illustrated in FIG. 4) which is designed as an inner telescope or inner part which fits within the socket or receptacle and which, for its part, is anchored on an implant 40 in the patient's jawbone.

The tooth base 34, i.e. the region of the tooth quadrant 16 covered by the prosthesis base material, surrounds the secondary structures 22 and 24. It extends downwards beyond the secondary structures 22 and 24 and forms a bearing element for receiving the secondary structures 22 and 24. Exemplified embodiments thereof are shown in FIGS. 2 to 7.

FIG. 2 illustrates a secondary structure 22 which is surrounded and enclosed by tooth material on three sides, i.e. on the occlusal side 26, the oral side 28 and the vestibular side 30. The tooth material lies against a large area of the secondary structure 22. The secondary structure 22 is glued at this point into the dental prosthesis 10 in a manner known per se, and the forces introduced are absorbed completely or almost completely by the tooth quadrant 16. In this respect, the tooth quadrant 16 forms the shape of a downwardly open U at this point.

However, the gum side legs of the secondary structure 22 are covered by the material of the prosthesis base 12. This material has a greater elasticity and is softer so that it is more suitable for lying against the alveolar crest. It surrounds the region of the tooth base 34 completely.

A modified embodiment of a dental prosthesis 10 in accordance with the invention in cross-section is illustrated in FIG. 3. In this case, as also in the remaining figures, like reference numerals indicate like or corresponding parts. In contrast to the solution shown in FIG. 2, the secondary structure 22 is covered on all sides at this location by a thin layer of base material 12.

It is received by the tooth base 34 which supports, in turn, the occlusal side 26, the oral side 28 and the vestibular side 30 of the secondary structure 22 in this respect via the layer of base material 12.

FIG. 4 shows a modified embodiment of the dental prosthesis 10, wherein a primary structure 38 is also illustrated in addition to the secondary structure 22.

The primary structure 38 is anchored via an implant 40 in the jawbone which is received by the alveolar crest 42. The prosthesis base material 12 lies in a planar manner on the alveolar crest 42 and thus permits tilt-free bearing of the prosthesis 10.

Also, in the case of the embodiment shown in FIG. 4, the prosthesis base material 12 extends as far as underneath the tooth base 34 and covers the secondary structure 22.

FIG. 5 shows a further embodiment of the dental prosthesis 10 in accordance with the invention. In the case of this solution, the tooth arrangement 14 is formed of two layers and consists of a dentine layer 44 and a tooth enamel layer 46.

The dentine layer 44 is covered completely by the tooth enamel 46 in the incisal region, as well as outside the prosthesis base 12, in a manner known per se. However, the dentine layer 44 extends in the form of a prolongation over the secondary structure 22.

In this respect, the tooth arrangement 14 also extends in the region of the tooth base 34 over the secondary structure 22. It supports the tooth quadrant 16 at this point and thus the dental prosthesis 10.

The dentine layer 44 is more opaque and the enamel material 46 more translucent in a manner known per se. In this case, the region of the tooth quadrant 16 is completely covered by the prosthesis base material 12.

Instead of a bearing on a secondary structure 22 which cooperates with a primary structure 38, it is also possible to provide a bearing of the prosthesis base 12 and thus of the tooth quadrant 16 on an abutment 50. In turn, the abutment 50 is mounted on the implant 40 in a manner known per se.

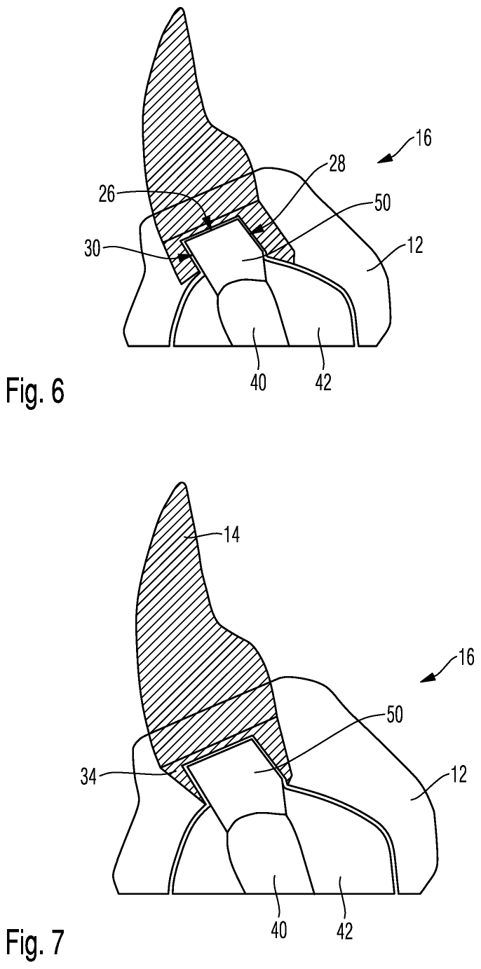

This solution is illustrated in FIG. 6. In turn, the tooth base 34 encloses the abutment 50 on the three sides 26, 28 and 30. In this case, it is also not necessary to produce an additional support structure in the form of a tertiary structure.

FIG. 7 illustrates a further configuration of an embodiment comprising the implant 40. In the case of this solution, the tooth quadrant 16 extends with the tooth base 34 in direct contact with the implant 40 and tapers downwards. In contrast to the embodiment shown in FIG. 6, this prevents direct contact between the alveolar crest 42 and the tooth quadrant 16.

In the case of this solution, the lateral support and thus the support taking the chewing movements into account is also provided as a matter of course.

1. A dental prosthesis comprising
a gum-coloured prosthesis base (12) and
a tooth-coloured tooth arrangement (14) which is partially embedded into the prosthesis base (12),
wherein the dental prosthesis is formed as a hybrid prosthesis,
wherein the prosthesis base has a contact surface which has a surface normal pointing towards the gums,
wherein the tooth arrangement (14) surrounds a secondary part (22, 24) or a primary part (38, 50) on at least two sides (26, 28, 30) and is formed as a one-piece tooth quadrant or as a segmented tooth quadrant (16).

2. The dental prosthesis as claimed in claim 1,
wherein the secondary part or the primary part supports the tooth arrangement and a layer of the base material lies therebetween.

3. The dental prosthesis as claimed in claim 1,
wherein the tooth-coloured tooth material of the tooth arrangement is fabricated of a material having a greater hardness than the hardness of the prosthesis material of the prosthesis base (12).

4. The dental prosthesis as claimed in claim 1,
wherein the secondary part is formed having an external telescope configuration and/or the primary part is formed as an abutment or as a tooth stump, which fits within the external telescope configuration.

5. The dental prosthesis as claimed in claim 1,
wherein the tooth arrangement (14) is produced by a machining or constructive method.

6. The dental prosthesis as claimed in claim 4,
wherein the tooth arrangement (14) surrounds, on its basal side, the external telescope configuration or the primary part in the oral, vestibular and occlusal directions.

7. The dental prosthesis as claimed in claim 1,
wherein the tooth arrangement (14) forms, on its basal side, which is the side facing the gum, a bearing element which receives the secondary part or the primary part, optionally via a layer of base material.

8. The dental prosthesis as claimed in claim 7,
wherein the bearing element of the tooth arrangement is embedded towards the outside in the vestibular and oral directions, into the prosthesis base material (12) which supports the bearing element from the outside, and the bearing element is exposed and open, on the inside towards the gums.

9. The dental prosthesis as claimed in claim 1,
wherein the tooth arrangement (14) is fabricated at least partially of a highly cross-linked polymethyl methacrylate (PMMA), and the prosthesis base (12) is fabricated of a less highly cross-linked PMMA in comparison to the highly cross-linked PMMA material used for the tooth arrangement.

10. The dental prosthesis as claimed in claim 7,
wherein the tooth arrangement (14) is constructed of two layers or multiple layers of dentine material and of enamel material which enamel material is more translucent than the dentine material and which dentine material extends in one piece to the bearing element to form the bearing element.

11. The dental prosthesis as claimed in claim 10,
wherein the tooth arrangement (14) has segments of at least 3 teeth which are fixedly connected to one another and to the bearing element via dentine material.

12. The dental prosthesis as claimed in claim 1,
wherein the dental prosthesis (10) is metal-free and is produced by machining or constructive methods.

13. The dental prosthesis as claimed in claim 7,
wherein the bearing element is formed from tooth material in an arcuate manner in plan view, corresponding to the tooth quadrant (16), and in a U-shape in cross-section, for the lower jaw prosthesis in the form of an upside-down U.

14. The dental prosthesis as claimed in claim 3,
wherein the tooth material extends to the contact surface of the prosthesis base and is covered by base material forming said contact surface.

15. The dental prosthesis as claimed in claim 3,
wherein the tooth material is covered by a layer of base material, in a layer thickness of at least 0.5 mm.

16. The dental prosthesis as claimed in claim 3,
wherein the tooth quadrant (16) and the prosthesis base (12) are each produced separately by milling from blanks or by an additive method and are fixedly connected to one another by an adhesive.

17. The dental prosthesis as claimed in claim 16,
wherein the adhesive comprises a self-polymerising adhesive.

18. The dental prosthesis as claimed in claim 9,
wherein the highly cross-linked PMMA comprises a double cross-linked (DCL) material.

19. The dental prosthesis as claimed in claim 15,
wherein the tooth material is covered by a layer of base material, in a layer thickness of at least about 0.8 mm.
